(19) United States
(12) Patent Application Publication
(10) Pub. No.: US 2011/0009714 A1
(43) Pub. Date: Jan. 13, 2011

(54) HEMODYNAMIC MONITORS AND ALARMS

(75) Inventor: Wei Zong, Arlington, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/919,819

(22) PCT Filed: Feb. 2, 2009
(86) PCT No.: PCT/IB09/50416

§ 371 Date: Aug. 27, 2010

Related U.S. Application Data
(63) Continuation-in-part of application No. 61/031,708, filed on Feb. 27, 2008.

(51) Int. Cl. A61B 5/02

(52) U.S. Cl. 600/301

(57) ABSTRACT

A hemodynamic monitoring instrument includes a processor and an output device. The processor (30) is arranged to receive a physiological parameter indicative of heart rate and a physiological parameter indicative of arterial blood pressure and is configured to compute (50) a hemodynamic parameter correlating with systemic vascular resistance (SVR) based on the received physiological parameter indicative of heart rate and the received physiological parameter indicative of arterial blood pressure. The output device includes least one of: (i) a display (24) configured to display the computed hemodynamic parameter; and (ii) an alarm (32, 34) configured to generate a perceptible signal responsive to the computed hemodynamic parameter satisfying an alarm criterion (52). The processor in some embodiments computes the hemodynamic parameter using a fuzzy membership function representing the heuristic ‘quantitative ABP measure is low AND quantitative HR measure is slightly high or high’ OR ‘quantitative ABP measure is very low’.

The following relates to the medical arts. It finds application in medical monitoring, medical alarm systems, and the like, for use in hospitals, urgent care centers, nursing homes, assisted care facilities, home medical monitoring, and the like.

Vasoconstriction medications, also called “vasopressors” cause vascular constriction and consequent increase in the arterial blood pressure (ABP). Accordingly, administration of a vasopressor is a recommended remedial action when a patient is in an abnormal hemodynamic state due to vascular resistance problems. Timely detection or prediction of the need for vasopressor intervention is crucial, because the time frame for intervention is relatively short and vital organs such as the brain can suffer irreversible damage leading to permanent debility or death if the vasopressor intervention is delayed.

To assess the desirability of vasopressor intervention, it is known that one should measure the mean aortic pressure (MAP), the cardiac output (CO), and the central venous pressure (CVP). These three quantities enable determination of the systemic vascular resistance (SVR) according to the relation MAP=(CO×SVR)+CVP and vasopressor intervention is indicated if the SVR falls below a threshold value.

In practice, measurement of central venous pressure is very invasive, so this quantity is generally assumed to have a negligible effect on the SVR. Measurement of cardiac output is also highly invasive, and CO measurements do not provide a continuously generated CO value that can be conveniently monitored. Unfortunately, CO is usually not negligible in determining SVR.

Measurement of the mean aortic pressure (MAP) is also very invasive. However, the arterial blood pressure (ABP) is readily measured in a non-invasive or minimally invasive manner, for example using a sphygmometer or an arterial line. The mean value of the ABP also approximately equals the MAP. In view of the invasiveness of the MAP, CO, and CVP measurements, it is commonplace for only ABP to be measured.

As a consequence, medical personnel are left to make the determination as to whether a vasopressor should be administered based on incomplete information. Under these circumstances, different physicians can make different qualitative judgments in deciding if and when the need for vasopressor intervention arises, typically relying on the available ABP measurements, other measured physiological parameters, and other information such as the patient's overall appearance, pre-existing medical conditions, or so forth. This situation leads to a lack of uniformity in medical care and can result in unnecessary vasopressor administration, or conversely failure to administer a vasopressor that would have been medically beneficial.

The following provides a new and improved apparatuses and methods which overcome the above-referenced problems and others.

In accordance with one aspect, a hemodynamic monitoring instrument is disclosed, comprising: a processor arranged to receive a physiological parameter indicative of heart rate and a physiological parameter indicative of arterial blood pressure and configured to compute a hemodynamic parameter correlating with systemic vascular resistance (SVR) based on the received physiological parameter indicative of heart rate and the received physiological parameter indicative of arterial blood pressure; and an output device including least one of (i) a display configured to display the computed hemodynamic parameter and (ii) an alarm configured to generate a perceptible signal responsive to the computed hemodynamic parameter satisfying an alarm criterion.

In accordance with another aspect, a hemodynamic monitoring method is disclosed, comprising: computing a quantitative hemodynamic parameter that is (i) functionally dependent upon a quantitative heart rate (HR) measure and a quantitative arterial blood pressure (ABP) measure and (ii) correlates with systemic vascular resistance (SVR); and at least one of (i) displaying the quantitative hemodynamic parameter and (ii) generating a perceptible signal indicative of an abnormal hemodynamic condition conditional upon the computed hemodynamic parameter satisfying an alarm criterion.

In accordance with certain other disclosed aspects, a computer medium is disclosed storing instructions executable to control a computer and display to perform the method of the immediately preceding paragraph, and a hemodynamic monitoring device is disclosed including a display and a processor programmed to perform the method of the immediately preceding paragraph.

One advantage resides in providing an instrument for determining on a quantitative basis when vasopressor intervention is indicated.

Another advantage resides in providing an instrument capable of making a timely determination as to when vasopressor intervention is indicated.

Another advantage resides in quantitatively monitoring a hemodynamic parameter closely related to systemic vascular resistance without resort to highly invasive measurement of the cardiac output.

Still further advantages of the present invention will be appreciated to those of ordinary skill in the art upon reading and understand the following detailed description.

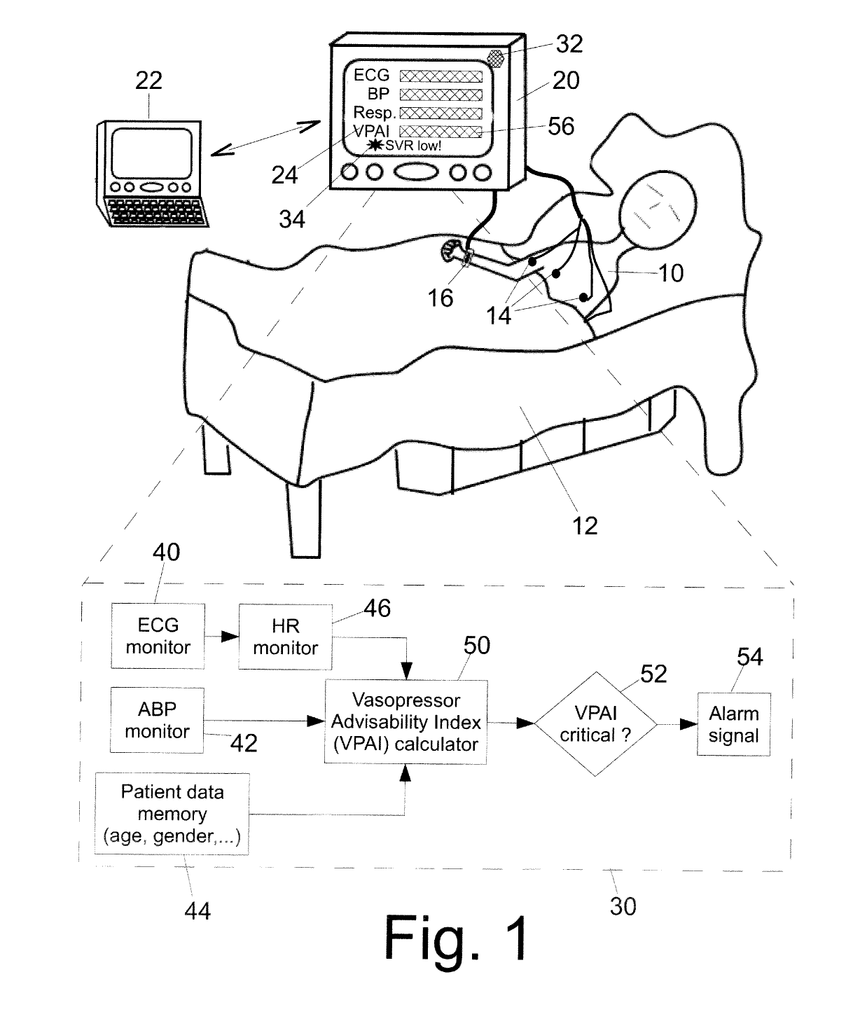

FIG. 1 diagrammatically shows a medical environment including a patient monitor including a hemodynamic monitoring instrument configured to indicate when vasopressor intervention is appropriate.

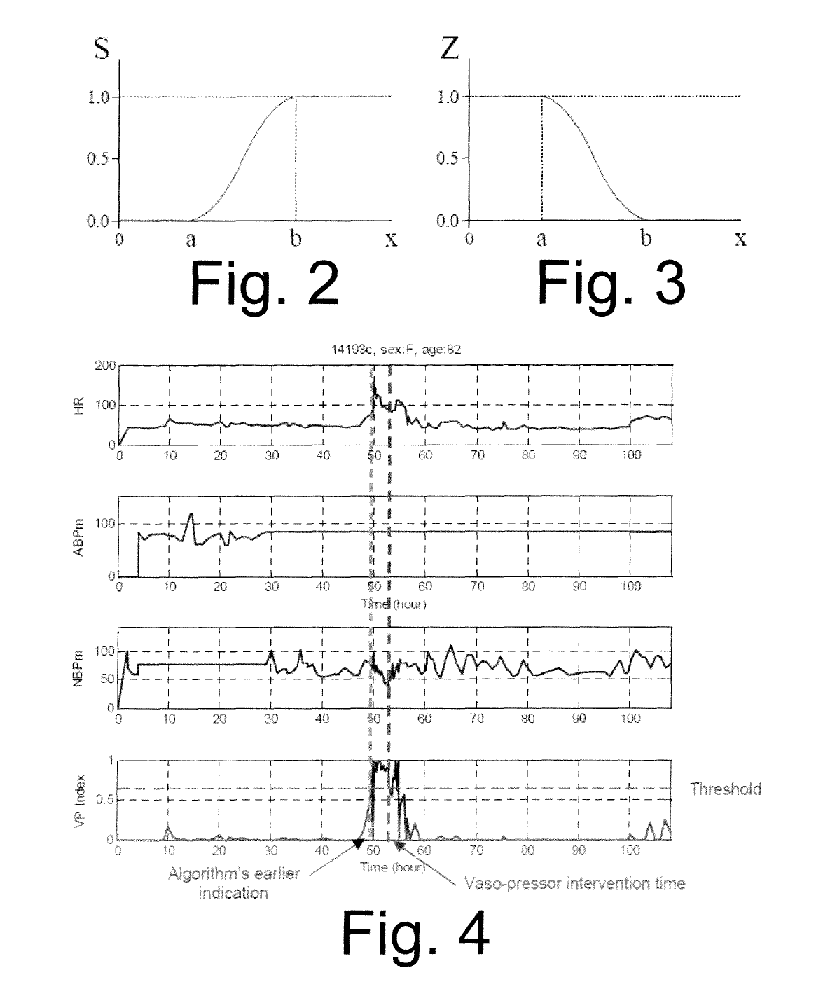

FIGS. 2 and 3 plot the shapes of the S-function and Z-function, respectively, used in the hemodynamic parameter computation implemented by the hemodynamic monitoring instrument of FIG. 1.

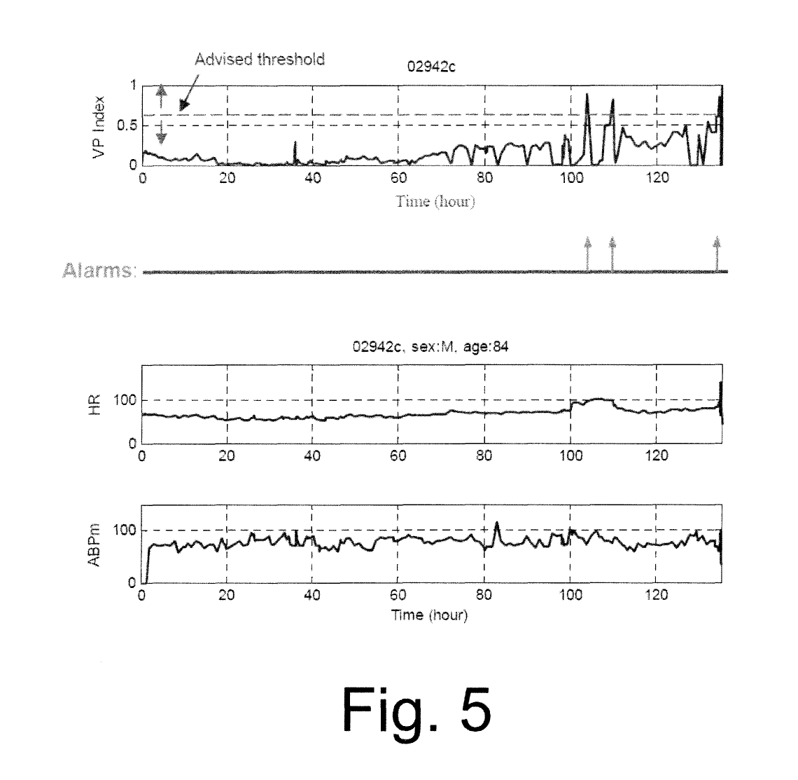

FIGS. 4 and 5 diagrammatically show illustrative outputs of embodiments of the patient monitor of FIG. 1 including the output of the hemodynamic monitoring instrument.

The hemodynamic monitors and alarms disclosed herein are based on some physiological insights. Starting with the relationship MAP=(CO×SVR)+CVP, if the central venous pressure (CVP) is assumed to be negligible, that is, CVP is approximated as zero, and mean arterial blood pressure (APB) is used as a substitute for the mean aortic pressure (MAP), then the systemic vascular resistance (SVR) can be approximated as SVR=ABP/CO. The cardiac output (CO) is equal to the cardiac stroke volume (SV) times the heart rate (HR), that is, CO=SV×HR. Physiologically, any variation in the cardiac output (CO) is typically due primarily to variation in the heart rate (HR), as the stroke volume (SV) is relatively constant for most patients and under most physiological conditions. Thus, CO∝HR captures the variability of the cardiac output (CO). Inserting this relationship into the expression for systemic vascular resistance (SVR) yields the relationship SVR∝ABP/HR. In other words, systemic vascular resistance (SVR) correlates with the hemodynamic parameter ABP/HR or with hemodynamic parameters proportional to or otherwise correlating with this ratio.

Vasopressor intervention is indicated if the systemic vascular resistance (SVR) is abnormally low, since the vasopressor intervention is intended to cause vascular constriction so as to increase the systemic vascular resistance (SVR). Based on the relationship SVR∝ABP/HR, one can predict that the systemic vascular resistance (SVR) will be low if the arterial blood pressure (APB) is low and the heart rate (HR) is high or slightly high. Since the heart rate (HR) cannot decrease too far (for example, HR does not fall below about 50 beats per minute for most people) one can also predict that the systemic vascular resistance (SVR) will be low if the arterial blood pressure (APB) is very low, regardless of the heart rate (HR) value.

On the other hand, if the heart rate (HR) is low, then the systemic vascular resistance (SVR) will not be abnormally low unless the arterial blood pressure (APB) is very low. A low (but not very low) arterial blood pressure (APB) coupled with a low heart rate (HR) is a normal condition which can arise during sleep, sedation, or other restful states. As a result, relying only upon the measured arterial blood pressure (APB) to determine when vasopressor intervention is indicated can result in false alarms caused by the patient entering a normal restful state during which both APB and HR decrease.

In view of the foregoing insights, a heuristic of the form: “ABP is low AND HR is slightly high or high” OR “ABP is very low” can be used to qualitatively estimate when vasopressure intervention is indicated. Such a qualitative heuristic alone is unfortunately not useful as a hemodynamic monitor or alarm. As disclosed herein, however, this heuristic can be quantified so as to provide a suitable basis for a hemodynamic monitor or alarm.

With reference to FIG. 1, a patient 10 is shown lying in a bed 12 such as is a typical situation in a hospital, emergency room, intensive care unit (ICU), cardiac care unit (CCU), or so forth. Depending upon the patient's condition, it is also contemplated that the patient 10 may be ambulatory, residing in a wheel chair, seated in a chair, or so forth. The patient is monitored by various medical monitoring devices, including in the illustrated embodiment an electrocardiographic (ECG) instrument with ECG electrodes 14, and a blood pressure monitor 16, which may for example be a wholly non-invasive sphygmometer or a minimally invasive arterial line. The illustrated blood pressure monitor 16 is wrist-based; however, a blood pressure monitor located on the upper arm or elsewhere on the patient 10 is also contemplated. If an arterial line is used to measure blood pressure, it may optionally be incorporated into an intravenous fluid delivery line or the like. The ECG and ABP monitors 14, 16 further include associated electronics for generating and optionally performing signal processing on ECG and blood pressure signals. In the illustrated embodiment, these electronics are embodied as a unitary multi-functional patient monitor 20 that provides the electronics for both ECG and ABP monitoring, as well as optionally providing the electronics for monitoring selected other physiological parameters such as respiration rate based on suitable physiological input signals. The multi-functional patient monitor 20 is optionally locally programmable using a built-in keypad or other built-in devices (not shown), and is additionally or alternatively remotely programmable using a computer 22 or other remote device communicating with the multi-functional patient monitor 20 using a wired or wireless digital communication pathway such as a wired or wireless local area network (LAN or WLAN), bluetooth wireless connection, or so forth. The illustrated multi-functional patient monitor 20 includes a display 24 that displays measured physiological parameters such as the ECG trace, blood pressure (BP) data, respiratory data, or so forth. The display can display these parameters in various ways, such as by current numerical value, by a trace showing parameter value as a function of time, or so forth.

The multi-functional patient monitor 20, together with the ECG and blood pressure monitoring instruments, also define a hemodynamic monitor and alarm instrument. This is illustrated in FIG. 1 by a diagrammatic processor 30 which is embodied by electronics of the multi-functional patient monitor 20. For example, the processor 30 is suitably a processor of the patient monitor 20 executing suitable software that performs processing implementing the hemodynamic instrument, displays the resulting hemodynamic parameter on the display 24, and generates an audible alarm output by an audio speaker 32, or a visual alarm 34 displayed on the display 24, or generates another perceptible alarm signal. While described with this physical construction as an illustrative example, it is to be understood that the hemodynamic instrument embodiments disclosed herein can be variously physically embodied, for example as a stand-alone instrument including blood pressure and heart rate monitoring capability, or as software running on a computer or other digital device at a nurses' station, or as a pocketable or wearable portable unit, or so forth. Moreover, the hemodynamic monitoring techniques disclosed herein can also be embodied as a digital storage medium such as for example, a magnetic disk, an optical disk, an Internet server, a random access memory (RAM), a read-only memory (ROM), or so forth, that stores instructions executable by the processor 30 or by another processor to perform the hemodynamic monitoring techniques disclosed herein.

The processor 30 suitably implements ECG monitoring 40 to receive and optionally perform signal processing of the ECG signal, and further implements arterial blood pressure (ABP) monitoring 42 to receive and optionally perform signal processing of the APB signal. In the illustrated embodiment, such monitor processing 40, 42 are suitably performed by the processor 30 executing software. Alternatively, these signals may be received from elsewhere, such as from an independent ECG monitor or an independent blood pressure monitor. The processor 30 may also include or have access to a memory 44 of the patient monitor 20 storing patient information such as patient age, patient gender or sex, or so forth. Heart rate monitor processing 46 performed by the processor 30 executing suitable software extracts the heart rate as a function of time from the ECG signal.

More generally, the hemodynamic instrument receives a physiological parameter indicative of heart rate and a physiological parameter indicative of arterial blood pressure. In the illustrated embodiment, the physiological parameter indicative of heart rate is the ECG signal and the output of the heart rate monitor instrument 46 is a heart rate indicated by the physiological parameter. However, another physiological parameter indicative of heart rate could be used, such as for example the output of a fingertip SpO₂ monitor, and a suitable processing unit would then produce the heart rate indicated by the physiological parameter by suitable processing of the fingertip SpO₂ monitor signal. Similarly, in the illustrated embodiment the physiological parameter indicative of arterial blood pressure is the output of the blood pressure monitoring instrument 16, 42 which performs suitable processing of the physiological signal generated by the blood pressure monitor 16 so as to output a mean arterial blood pressure (ABP) indicated by the blood pressure monitor signal. Again, another physiological parameter indicative of mean arterial blood pressure (ABP) could be used, optionally with suitable processing to derive the ABP signal indicated by such other physiological parameter indicative of mean arterial blood pressure (ABP).

The information relevant for estimating a hemodynamic parameter correlating with systemic vascular resistance (SVR) includes: (i) the heart rate (HR) indicated by the physiological parameter indicative of heart rate; (ii) the mean arterial blood pressure (ABP) indicated by the physiological parameter indicative of arterial blood pressure; and (iii) optionally other patient data such as patient age or patient gender or sex. This information is input to a Vasopressor Advisability Index (VPAI) calculator 50, which computes a hemodynamic parameter quantifying the heuristic “ABP is low AND HR is slightly high or high” OR “ABP is very low” where APB denotes an arterial blood pressure indicated by the physiological parameter indicative of arterial blood pressure and HR denotes a heart rate indicated by the physiological parameter indicative of heart rate.

The hemodynamic parameter computed by the VPAI calculator 50 is denoted herein as the vasopressor advisability index (VPAI), and provides a quantitative hemodynamic parameter that is (i) functionally dependent upon the quantitative heart rate (HR) measure and the quantitative mean arterial blood pressure (APB) measure and (ii) correlates with systemic vascular resistance (SVR). The VPAI is compared with a criticality criterion by a comparator 52, and if the VPAI satisfies the criticality criterion then an alarm signal 54 is generated as a perceptible signal such as an audible alarm output by the speaker 32 or a visual alarm 34 displayed on the display 24. Additionally or alternatively to generating the alarm signal 54, a VPAI display 56 may be presented on the display 24 of the patient monitor 20 in the form of a plot of VPAI value versus time, or as a numerical display of the current VPAI value, or as a combination of a plot and current value numerical display, or in another suitable form. The provided VPAI information 54, 56 provides an objective and quantitative basis upon which a physician or other medical personnel can assess the advisability of administering vasopressor intervention.

Having described some embodiments of the hemodynamic monitoring instrument will illustrative reference to the illustrated embodiment of FIG. 1, some preferred formulations of the VPAI calculation are now set forth. The function of the VPNI calculation is to quantitatively capture the pattern: “ABP is low and HR is slightly high or high” OR “ABP is very low” as an index or other quantitative value. In some preferred formulations, the VPNI is computed using fuzzy logic. In an illustrative formulation, three fuzzy variables are defined to quantitatively represent the three constituent heuristics “HR is high or slightly high”, “ABP is low”, and “ABP is very low”.

With reference to FIGS. 2 and 3, in one suitable formulation the three constituent heuristics are represented by fuzzy variables having the shape of an S-function shown in FIG. 2, or having the shape of a Z-function shown in FIG. 3. Quantitatively, these functions are set forth as follows:

$$\begin{matrix}S ( x ; a , b ) = \{ \begin{matrix}0 , & x \leq a \\ 2 {( \frac{x - a}{b - a} )}^{2} , & a < x \leq \frac{a + b}{2} \\ 1 - 2 {( \frac{x - b}{b - a} )}^{2} , & \frac{a + b}{2} < x \leq b \\ 1 , & b < x ,\end{matrix} \\ and & ( 1 ) \\ Z ( x ; a , b ) = 1 - S ( x ; a , b ) . & ( 2 )\end{matrix}$$

Using these functions, the heuristic “HR is slightly high or high” is quantified as:

μ_{HR}_{_{—}}ᵢₛ_{_{—}}_{slightlyHigh}_{_{—}}ₒᵣ_{_{—}}_{high}=S(HR; 70+AF_{HR}+SF_{HR}, 120+AF_{HR}+SF_{HR}) (3),

where HR is the heart rate in beats per minute indicated by the physiological parameter indicative of heart rate, AF_{HR} is an optional patient age adjustment factor, SF_{HR} is an optional patient gender or sex adjustment factor, the S-function low-end boundary a is 70+AF_{HR}+SF_{HR}, and the S-function high-end boundary b is 120+AF_{HR}+SF_{HR}.

In similar fashion, quantitative fuzzy variables may be defined for the remaining constituent heuristics as follows:

μ_{APB}_{_{—}}ᵢₛ_{_{—}}_{low}=Z(ABP; 60+AF1_{ABP}+SF1_{ABP}, 80+AF1_{ABP}+SF1_{ABP}) (4),

and

μ_{APB}_{_{—}}ᵢₛ_{_{—}}_{veryLow}=Z(ABP; 40+AF2_{ABP}+SF2_{ABP}, 60+AF2_{ABP}+SF2_{ABP}) (5)

where ABP is the mean arterial blood pressure in mmHg indicated by the physiological parameter indicative of arterial blood pressure, and AF1_{ABP} and AF2_{ABP} are optional patient age adjustment factors, and SF1_{ABP} and SF2_{ABP} are optional patient gender or sex adjustment factors.

The optional adjustment factors AF_{HR}, AF1_{ABP}, AF2_{ABP}, SF_{HR}, SF1_{ABP}, and SF2_{ABP} are suitably derived from patient data compilations representing typical heart rates and mean arterial blood pressure values as a function of patient age and patient gender or sex. For example, it is known that the aterial blood pressure tends to increase monotonically with patient age, so the optional adjustment factor AF1_{ABP} is suitably a monotonically increasing function of patient age reflecting this known trend. It is contemplated for some of these adjustment factors to have negative values. Adjustment factors for patient age and patient gender or sex are expressly set forth herein as illustrative examples. However, it is contemplated that adjustment factors for other patient characteristics may be incorporated. Additionally or alternatively, it is contemplated to have patient-specific boundaries for the fuzzy variables, for example based on actually recorded heart rate values provided in the medical history of a specific patient.

The quantitative fuzzy variable set forth in each of Equations (3), (4), and (5) have output values in the range [0,1] due to the limits set by the S- and Z-functions. For μ_{HR}_{_{—}}ᵢₛ_{_{—}}_{slightlyHigh}_{_{—}}ₒᵣ_{_{—}}_{high} higher values indicate higher agreement with the heuristic “Heart rate (HR) is slightly high or high”. For μ_{APB}_{_{—}}ᵢₛ_{_{—}}_{low}, higher values indicate higher agreement with the heuristic “Mean arterial blood pressure (APB) is low”. For μ_{APB}_{_{—}}ᵢₛ_{_{—}}_{veryLow}, higher values indicate higher agreement with the heuristic “Mean arterial blood pressure (APB) is very low”. The vasopressor advisability index, denoted μ_{VPAI}, is suitably defined as a fuzzy composite variable according to:

μ_{VPAI}=(μ_{APB}_{_{—}}ᵢₛ_{_{—}}_{low} [symbol] μ_{HR}_{_{—}}ᵢₛ_{_{—}}_{slightlyHigh}_{_{—}}ₒᵣ_{_{—}}_{high}) [symbol] μ_{APB}_{_{—}}ᵢₛ_{_{—}}_{veryLow} (6),

where the symbol [symbol] denotes the standard fuzzy intersection, defined as μ_{A}(x) [symbol] μ_{B}(x)=min{μ_{A}(x), μ_{B}(x)}, and the symbol [symbol] denotes the standard fuzzy union, defined as μ_{A}(x) [symbol] μ_{B}(x)=max{μ_{A}(x), μ_{B}(x)}. The vasopressor adviseability index (μ_{VPAI}) is also bounded to lie in the range [0,1], with higher values indicating higher agreement with the heuristic “Vasopressor intervention is advisable”.

The criticality criterion implemented by the comparator 52 can be constructed in various ways. One suitable criterion is:

IF (μ_{VPAI}>Iₜₕ) THEN Vasopressor intervention advised (7)

where Iₜₕ is a threshold value. The criticality criterion can also be expressed using a fuzzy conditional statement according to:

IF (μ_{APB}_{_{—}}ᵢₛ_{_{—}}_{low} AND μ_{HR}_{_{—}}ᵢₛ_{_{—}}_{slightlyHigh}_{_{—}}ₒᵣ_{_{—}}_{high}) OR μ_{APB}_{_{—}}ᵢₛ_{_{—}}_{veryLow} THEN Vasopressor intervention advised (8).

In Equation (8), the first part (μ_{APB}_{_{—}}ᵢₛ_{_{—low}} AND μ_{HR}_{_{—}}ᵢₛ_{_{slightlyHigh}}_{_{—}}ₒᵣ_{_{—}}_{high}) identifies abnormal events when the blood pressure drops noticeably while the heart rate is faster than usual. The second part μ_{APB}_{_{—}}ᵢₛ_{_{—}}_{veryLow} identifies the situation in which the blood pressure decreases to a critically low value, regardless of the heart rate. The threshold for activating the alarm (e.g., the threshold Iₜₕ of Equation (7)) is suitably obtained empirically by analysis of reference patient databases, or may also be determined by clinical users according to specific patient situations. In some embodiments, the hemodynamic monitoring instrument has a user-selectable threshold that is adjustable.

To summarize, the hemodynamic monitoring instrument operates in the following way. For each time point, the instrument receives a heart rate (HR) value indicated by the physiological parameter indicative of heart rate, a mean arterial blood pressure (APB) value indicated by the physiological parameter indicative of arterial blood pressure, and optionally receives further relevant information such as patient age, patient gender or sex, or so forth, and generates a vasopressor advisability index (μ_{VPAI}) according to Equation (6). The vasopressor adviseability index (μ_{VPAI}) is displayed in real-time, optionally along with the heart rate HR and ABP values, and is compared to a threshold or other criticality criterion. If the vasopressor advisability index (μ_{VPAI}) satisfies the criticality criterion, then a vasopressor intervention advisability alarm is issued which indicates advisability of vaso-pressor intervention or another remediation of the abnormal systemic vascular resistance (SVR) condition.

In the following, some illustrative examples of operation of a hemodynamic monitoring instrument constructed in substantial accordance with the teachings set forth herein are set forth.

With reference to FIG. 4, an ICU record is shown for an 82-year old female patient in the MIMIC-II database. See Saeed et al., “MIMIC-II: A massive temporal ICU patient database to support intelligent patient monitoring”, Computers in Cardiology 2002; 29:641-44. The panels of FIG. 4 plot the patient heart rate (HR) measurements, patient mean arterial blood pressure (ABPm) measurements, and mean non-invasive blood pressure (NBPm) measurements. Note that the flat-line periods in the ABPm and NBPm traces refer to time intervals during which the measurements were not taken or were not available in the MIMIC-II database. The bottom panel plots the vasopressor advisability index (μ_{VPAI}, denoted “VP Index” in the relevant panel of FIG. 4) computed for those time intervals during which either APBm or NBPm values were available. In the bottom panel, the threshold Iₜₕ of Equation (7) is indicated by a horizontal dashed line.

As seen in FIG. 4, the patient's vasopressor advisability index (μ_{VPAI}) value became high (exceeding a preset threshold of about 0.6) around at 50 hours, this point in time being indicated by the lefthand vertical dashed line in FIG. 4, indicating that there was a critical event appearing corresponding to a low systemic vascular resistance (SVR). However, the patient's medication record stored in the MIMIC-II Database shows that an actual vasopressor (Neosynephrine) intervention was applied to the patient at around 53 hours, this point in time being indicated by the righthand vertical dashed line in FIG. 4. This is about 2.5 hours after the vasopressor advisability index (μ_{VPAI}) advised to initiate vasopressor intervention. The vasopressor intervention was successful, as indicated by a substantial decrease in the vasopressor advisability index (μ_{VPAI}) shortly after initiation of the vasopressor intervention.

As further seen in FIG. 4, the NBPm also dropped to a noticeable low level at times other than around the 50 hour interval indicated by the vasopressor advisability index (μ_{VPAI}) as corresponding to an event calling for vasopressor intervention. Such noticeable decreases in the NBPm occurred, for example, in the 38-48 hour interval, in the 60-70 hour interval, and in the 80-90 hour interval. However, the vasopressor advisability index (μ_{VPAI}) did not produce high values in those periods, indicating that no false alarms were triggered by these blood pressure decreases. In contrast, a physician monitoring the blood pressure without having the benefit of the vasopressor advisability index (μ_{VPAI}) might have elected to initiate (unnecessary) vasopressor intervention at one or more of these time intervals.

With reference to FIG. 5, another illustrative example is shown, in which the vasopressor advisability index (μ_{VPAI}) is computed for patient monitoring data of an 84 year old male patient, again taken from the MIMIC-II database. The vasopressor advisability index (μ_{VPAI}) values are computed as a function of time with adjustments for the patient's age and gender, using the heart rate (HR) and mean arterial blood pressure (ABP) measurements stored in the MIMIC-II database. The vasopressor advisability index (μ_{VPAI}, denoted in FIG. 5 as “VP Index”) is plotted in the top panel of FIG. 5. The threshold Iₜₕ of Equation (7) is indicated by a horizontal dashed line in FIG. 5. When the vasopressor advisability index exceeds the threshold Iₜₕ, a critical event is detected and a perceptible alarm is generated to notify the physician or other medical personnel that vasopressor intervention is deemed to be advisable. Upon perceiving the alarm, the physician or other medical personnel can review the current and recent physiological data (for example using the patient monitor 20 of FIG. 1) to confirm the occurrence of an abnormal hemodynamic event indicative of a low systemic vascular resistance. This confirmation may entail, for example, consideration of the heart rate (HR) and mean arterial blood pressure (ABPm) readings shown in FIG. 5 and suitably plotted along with the vasopressor advisability index on the display 24 of the patient monitor 20.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A hemodynamic monitoring instrument comprising:
a processor arranged to receive a physiological parameter indicative of heart rate and a physiological parameter indicative of arterial blood pressure and configured to compute a hemodynamic parameter correlating with systemic vascular resistance (SVR) based on the received physiological parameter indicative of heart rate and the received physiological parameter indicative of arterial blood pressure, the hemodynamic parameter correlating with SVR quantifying the heuristic “ABP is low AND HR is slightly high or high” OR “ABP is very low” where APB denotes an arterial blood pressure indicated by the physiological parameter indicative of arterial blood pressure and HR denotes a heart rate indicated by the physiological parameter indicative of heart rate; and
an output device including least one of (i) a display configured to display the computed hemodynamic parameter and (ii) an alarm configured to generate a perceptible signal responsive to the computed hemodynamic parameter satisfying an alarm criterion.

2. The hemodynamic monitoring instrument as set forth in claim 1, wherein the instrument is integral with a patient monitor configured to display at least one of (i) a heart rate derived from the physiological parameter indicative of heart rate and (ii) an arterial blood pressure derived from the physiological parameter indicative of arterial blood pressure.

3. The hemodynamic monitoring instrument as set forth in claim 1, further comprising:
a memory storing a patient age, the processor configured to compute the hemodynamic parameter substantially correlating with SVR further based on the stored patient age.

4. The hemodynamic monitoring instrument as set forth in claim 1, further comprising:
a memory storing a patient gender, the processor configured to compute the hemodynamic parameter substantially correlating with SVR further based on the stored patient gender.

5. The hemodynamic monitoring instrument as set forth in claim 1, further comprising:
a memory storing a patient age and a patient gender, the processor configured to compute the hemodynamic parameter substantially correlating with SVR further based on the stored patient age and stored patient gender.

6. The hemodynamic monitoring instrument as set forth in claim 1, wherein the processor is configured to compute the hemodynamic parameter substantially correlating with SVR based on:
a fuzzy membership function indicative of whether a heart rate indicated by the physiological parameter indicative of heart rate is slightly high or high,
a fuzzy membership function indicative of whether an arterial blood pressure indicated by the physiological parameter indicative of arterial blood pressure is low, and
a fuzzy membership function indicative of whether the arterial blood pressure indicated by the physiological parameter indicative of arterial blood pressure is very low.

7. The hemodynamic monitoring instrument as set forth in claim 1, wherein the processor is configured to compute the hemodynamic parameter substantially correlating with SVR based on:
a first term defined by conjunctive combination of a first sub-term indicative of whether a heart rate indicated by the physiological parameter indicative of heart rate is slightly high or high and a second sub-term indicative of whether an arterial blood pressure indicated by the physiological parameter indicative of arterial blood pressure is low, and
a second term indicative of whether the arterial blood pressure indicated by the physiological parameter indicative of arterial blood pressure is very low.

8. (canceled)

9. The hemodynamic monitoring instrument as set forth in claim 1, wherein the processor is configured to increase HR by AF_{HR} where AF_{HR} denotes a correction term based on patient age.

10. The hemodynamic monitoring instrument as set forth in claim 1, wherein the processor is configured to increase ABP by AF_{ABP} where AF_{ABP} denotes a correction term based on patient age that is monotonically increasing with patient age.

11. The hemodynamic monitoring instrument as set forth in claim 1, wherein the output device comprises:
a display configured to plot the computed hemodynamic parameter as a function of time.

12. A hemodynamic monitoring method comprising:
computing a quantitative hemodynamic parameter that is (i) functionally dependent upon a quantitative heart rate (HR) measure and a quantitative arterial blood pressure (ABP) measure and (ii) correlates with systemic vascular resistance (SVR) and (iii) quantifies the heuristic “quantitative ABP measure is low AND quantitative HR measure is slightly high or high “OR” quantitative ABP measure is very low”; and
at least one of (i) displaying the quantitative hemodynamic parameter and (ii) generating a perceptible signal indicative of an abnormal hemodynamic condition conditional upon the computed hemodynamic parameter satisfying an alarm criterion.

13. The hemodynamic monitoring method as set forth in claim 12, further comprising:
determining the quantitative HR measure;
determining the quantitative ABP measure.

14. The hemodynamic monitoring method as set forth in claim 12, wherein the computing of the quantitative hemodynamic parameter is further functionally dependent upon at least one of a patient age and a patient gender.

15. The hemodynamic monitoring method as set forth in claim 12, wherein the computing of the quantitative hemodynamic parameter comprises:
computing a first fuzzy variable indicative of whether the quantitative HR measure is slightly high or high;
computing a second fuzzy variable indicative of whether the quantitative ABP measure is low; and
computing a third fuzzy variable indicative of whether the quantitative ABP measure is very low.

16. The hemodynamic monitoring method as set forth in claim 15, wherein the computing of the quantitative hemodynamic parameter further comprises:
computing a fuzzy composite variable combining the first, second, and third fuzzy variables using fuzzy combinational operators selected from the group consisting of fuzzy intersection and fuzzy union, the fuzzy composite variable correlating with systemic vascular resistance (SVR).

17. (canceled)

18. The hemodynamic monitoring instrument as set forth in claim 12, wherein computing of the quantitative hemodynamic parameter further comprises increasing the quantitative HR measure by AF_{HR} where AF_{HR} denotes a correction term based on patient age.

19. The hemodynamic monitoring instrument as set forth in claim 12, wherein computing of the quantitative hemodynamic parameter further comprises increasing the quantitative ABP measure by AF_{ABP} where AF_{ABP} denotes a correction term based on patient age that is monotonically increasing with patient age.

20. The hemodynamic monitoring instrument as set forth in claim 12, wherein computing of the quantitative hemodynamic parameter comprises:
computing a quantitative hemodynamic parameter having a value substantially equivalent to the result μ_{VPAI} of the equation:
μ_{VPAI}=(μ_{APB}_{_{—}}ᵢₛ_{_{—}}_{low} [symbol] _{μ}_{HR}_{_{—}}ᵢₛ_{_{—}}_{slightlyHigh}_{_{—}}ₒᵣ_{_{—}}_{high}) [symbol] μ_{APB}_{_{—}}ᵢₛ_{_{—}}_{veryLow}
where
μ_{HR}_{_{—}}ᵢₛ_{_{—}}_{slightlyHigh}_{_{—}}ₒᵣ_{_{—}}_{high}=S(HR; 70+F_{HR}, 120+F_{HR})
and
μ_{APB}_{_{—}}ᵢₛ_{_{—}}_{low}=Z(ABP; 60+F1_{ABP}, 80+F1_{ABP})
and
μ_{APB}_{_{—}}ᵢₛ_{_{—}}_{veryLow}=Z(ABP; 40+F2_{ABP}, 60+F2_{ABP})
and where HR denotes the quantitative HR measure, ABP denotes the quantitative ABP measure, and F_{HR}, F1_{ABP}, and F2_{ABP} denote patient-dependent adjustment factors.

21. A computer medium storing instructions executable to control a computer and display to perform the method of claim 12.

22. (canceled)

23. A hemodynamic monitoring device including a display and a processor programmed to perform the method of claim 12.
